(19) United States
(12) Patent Application Publication
(10) Pub. No.: US 2026/0041582 A1
(43) Pub. Date: Feb. 12, 2026

(54) APPARATUSES AND METHODS FOR CRYO-BASED ANESTHESIA

(71) Applicant: Varian Medical Systems, Inc., Palo Alto, CA (US)

(72) Inventors: Hongxuan Zhang, Austin, TX (US); Vineel Vallapureddy, Plymouth, MN (US); Ryan Medema, Georgetown, TX (US)

(21) Appl. No.: 18/800,871

(22) Filed: Aug. 12, 2024

(51) Int. Cl. A61F 7/00; G16H 40/63

(52) U.S. Cl. CPC A61F 7/007; G16H 40/63; A61F 2007/0055; A61F 2007/0056; A61F 2007/0093

(57) ABSTRACT

A cryo-treatment apparatus includes a cryoprobe assembly with a needle for insertion at a target tissue, a cryogen delivery apparatus fluidly connected to the cryoprobe assembly, and a cryo-treatment control apparatus comprising at least one processor and memory, wherein the cryo-treatment control apparatus is configured to: obtain cryoprobe operating information from one or more sensors positioned in the needle of the cryoprobe, the cryoprobe operating information characterizing one or more operating parameters of the cryoprobe, deliver a cryogen to the cryoprobe to produce a cryo-treatment zone in an area of the target tissue, the cryo-treatment zone having a treatment temperature range of about −50 degrees C. to about −100 degrees C., and maintain the cryo-treatment zone in the treatment temperature range for a predetermined period of time.

### FIELD

The present disclosure relates to apparatuses and methods for cryo-based anesthesia.

### BACKGROUND

This section provides background information related to the present disclosure which is not necessarily prior art.

Cryo-based anesthesia is a treatment whereby neurological tissues such as nerve tissue, spinal tissue, brain tissues, or other similar tissues of the nervous system may be subjected to cold temperatures. The low temperatures may interrupt and/or modulate transmission of nervous signals for the interruption or modulation of pain in a patient. Such exposure to cold temperatures is performed without contaminating or damaging the neurological tissues or the nervous system such that other functions of the neurological tissues can continue while interrupting or modulating the pain in the patient.

Systems and methods for providing cryo-treatment treatments may include cryoablation probes that are introduced at or near target tissue in a patient. A cryoablation system may include an extremely cold cryogen (liquid, gas, or mixed phase) that may be passed through a probe in thermal contact with the target tissue. Heat from the tissue passes from the tissue, through the probe, and into the cryogen that removes heat from the targeted tissue. This removal of heat causes tissue to freeze, resulting in the destruction of the targeted tissue. It is desirable that the cryogen is of sufficiently low temperature to quickly and efficiently cause the targeted tissues to freeze.

Traditional or existing cryoprobes and related methods cannot be used to perform cryo-based anesthesia because such apparatuses and methods result in the destruction of tissue rather than the interruption or modulation of pain. Traditional or existing cryoprobes may operate at temperatures too low to perform cryo-based anesthesia or may not be operated with sufficient controls to prevent damage to neurological tissues. There exists a need, therefore, for improved apparatuses, cryoprobes and related methods that efficiently and accurately achieve a desired temperature at the targeted neurological tissue while preventing or minimizing the risk of exposing the targeted neurological tissue to temperatures that may cause permanent damage.

### SUMMARY

This section provides a general summary of the disclosure, and is not a comprehensive disclosure of its full scope or all of its features.

In various embodiments of the present disclosure, improved cryo-based anesthesia apparatuses and methods are provided. The cryo-based anesthesia apparatuses and methods may allow freezing cycles to be performed with tightly controlled and efficient temperature ranges. Such temperature ranges may perform a modulation or blockage of pain and do not permanently damage targeted neurological tissues.

In some embodiments of the present disclosure, a cryo-treatment apparatus is provided. The cryo-treatment apparatus may include a cryoprobe assembly comprising a needle for insertion at a target tissue, a cryogen delivery apparatus fluidly connected to the cryoprobe assembly, and a cryo-treatment control apparatus comprising at least one processor and memory, wherein the cryo-treatment control apparatus is configured to obtain cryoprobe operating information from one or more sensors positioned in the needle of the cryoprobe. The cryoprobe operating information may characterize one or more operating parameters of the cryoprobe. The control apparatus may also be configured to deliver a cryogen to the cryoprobe to produce a cryo-treatment zone in an area of the target tissue. The cryo-treatment zone may have a treatment temperature range of about −50 degrees C. to about −100 degrees C. The control apparatus may also be configured to maintain the cryo-treatment zone in the treatment temperature range for a predetermined period of time.

In one aspect, the needle may define a Joule-Thompson expansion chamber at a distal end thereof.

In another aspect, the cryogen delivery apparatus may include a Dewar and a pump configured to deliver the cryogen to the cryoprobe assembly.

In another aspect, the cryo-treatment zone may be maintained in the treatment temperature range by adjusting a pressure of the cryogen delivered to the needle.

In another aspect, the cryogen comprises Argon.

In another aspect, the needle may include a heater and the cryo-treatment control apparatus is configured to energize and de-energize the heater to maintain the cryo-treatment zone in the treatment temperature range.

In another aspect, the cryogen comprises liquid Nitrogen.

In another aspect, the step of delivering the cryogen to the cryoprobe may include pulsing the cryogen.

In another aspect, the target tissue comprises a neurological tissue.

In another aspect, the cryo-treatment zone may change a function of the target tissue but does not permanently damage the target tissue.

In another aspect, the cryo-treatment zone may be maintained in the treatment temperature range by adjusting a pressure of the cryogen delivered to the needle and adjusting a power signal delivered to a heater in the needle.

In another aspect, the delivery of the cryogen to the cryoprobe causes an iceball to form at the target tissue, and a desired diameter of the iceball is less than 2 cm.

In another aspect, the needle may include a conductive pad coupled to a power source for electrical stimulation of target tissue.

In another aspect, the cryo-treatment control apparatus may include a trained machine learning model configured to control at least one of a supply valve, a supply pump, a heater in a Dewar, and a heater in the needle.

In another aspect, one or more sensors may be positioned on or in the heater in the needle.

In some embodiments, a method of performing cryo-based anesthesia is provided. The method may include obtaining, via a cryo-treatment control apparatus comprising at least one processor and memory, cryoprobe operating information from one or more sensors positioned in a needle of a cryoprobe, the cryoprobe operating information characterizing one or more operating parameters of the cryoprobe; delivering, via a cryogen delivery apparatus coupled to the cryo-treatment control apparatus, a cryogen to the cryoprobe to produce a cryo-treatment zone in an area of the target tissue, the cryo-treatment zone having a treatment temperature range of about −50 degrees C. to about −100 degrees C.; and maintaining, via the cryogen delivery apparatus and the cryo-treatment control apparatus, the cryo-treatment zone in the treatment temperature range for a predetermined period of time.

In one aspect, the needle may define a Joule-Thompson expansion chamber at a distal end thereof and the cryo-treatment zone is maintained in the treatment temperature range by adjusting a pressure of the cryogen delivered to the needle.

In another aspect, the cryogen delivery apparatus may include a Dewar and a pump configured to deliver liquid nitrogen to the cryoprobe assembly and the cryo-treatment zone is maintained in the treatment temperature range by adjusting a pressure of the cryogen delivered to the needle and adjusting a power signal delivered to a heater in the needle.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an illustration of an example cryo-based anesthesia apparatus in accordance with some embodiments of the present disclosure.

FIG. 2 is a block diagram illustrating an example cryo-based anesthesia apparatus in accordance with some embodiments of the present disclosure.

FIG. 3 is a diagram illustrating aspects of an example cryo-control model in accordance with some embodiments of the present disclosure.

FIG. 4 is a graph illustrating performance of an example cryo-based anesthesia method of the present disclosure.

FIG. 5 is a block diagram illustrating an example cryo-based anesthesia apparatus in accordance with some embodiments of the present disclosure.

FIG. 6 is a diagram illustrating aspects of an example cryo-control model in accordance with some embodiments of the present disclosure.

FIG. 7 is a graph illustrating performance of an example cryo-based anesthesia method of the present disclosure.

FIG. 8 is a flow chart illustrating an example method of performing cryo-based anesthesia in accordance with some embodiments of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In some embodiments of the present disclosure, a cryo-based anesthesia apparatus or cryo-treatment apparatus is provided. The cryo-based anesthesia apparatus may include a cryoprobe that includes a needle that can be positioned at or near a neurological target tissue in a patient. The cryo-based anesthesia apparatus may deliver a cryogen to the needle of the cryoprobe to lower the temperature of the neurological target tissue to a target temperature that interrupts or modulates transmission of the neurological tissue without causing permanent damage or contaminating the neurological tissue. Such a target temperature may be in a range of about −50 degrees Celsius to about −100 degrees Celsius.

In various embodiments described below, the cryo-based anesthesia apparatus may include a Joule-Thompson cryoprobe or a liquid Nitrogen cryoprobe to achieve the desired target temperatures. The temperature of the cryo-treatment zone may be controlled to remain in a target temperature range so as to remain below a temperature at which contamination or permanent damage may be caused to the neurological target tissue through the use of a cryo-treatment control apparatus. The cryo-treatment control apparatus may maintain the neurological target tissue at or in the target temperature range by controlling one or more of the pressure of the cryogen, a power signal of a heater in the cryoprobe, a flow rate of the cryogen, and/or a pulse profile of the cryogen. Feedback from one or more sensors positioned in the cryoprobe may supply cryoprobe operating information to the cryo-treatment control apparatus. The cryo-treatment control apparatus may adjust the operation of the cryo-based anesthesia apparatus to maintain the neurological target tissue at the target temperature range.

Traditional or existing cryoprobes and related apparatuses are typically used in the context of cryoablation. Existing cryoprobes do not operate at the higher temperatures required to perform cryo-based anesthesia nor do they operate to achieve limited size of an iceball that is created during the procedure. Existing cryoprobes, related apparatuses and related methods typically seek to achieve extremely low temperatures lower than −100 degrees Celsius as quickly as possible. Such apparatuses and methods are designed to destroy a target tissue. The apparatuses and methods of the present disclosure are improvements over existing apparatuses and methods because they can achieve the moderated temperatures used in cryo-based anesthesia to achieve modulated or limited transmission by the target neurological tissues without or with reduced risk of damage to the neurological tissue and/or surrounding tissues.

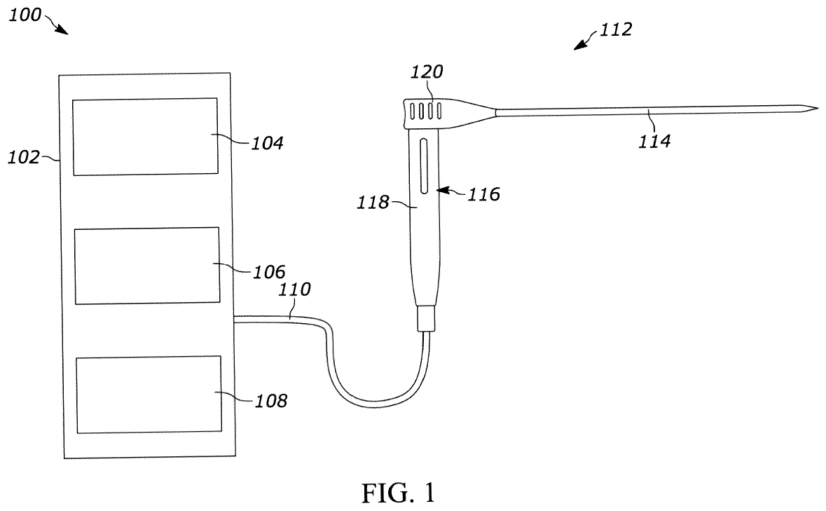

Referring now to FIG. 1, an example cryo-treatment apparatus 100 is shown. The cryo-treatment apparatus 100 may include a cryo-treatment console 102, and a cryoprobe assembly 112. The cryo-treatment console 102 may include a cryo-treatment control 104, a cryogen delivery apparatus 106 and a cryogen source 108. The cryo-treatment control 104 may include a computing device or other controller that can be used to control delivery of a cryogen (e.g., Argon, helium, Nitrogen, or the like) from the cryogen source 108 to the cryoprobe assembly 112 using the cryogen delivery apparatus 106. The cryogen source 108 may be a suitable Dewar or other container that can be filled with a cryogen. The cryogen delivery apparatus 106 may include a pump, one or more valves, and other suitable fluid delivery devices to fluidly connect the cryogen source to the cryogen line 110 of the cryoprobe assembly 112. Upon the initiation of a freezing cycle, the cryo-control 104 may cause the cryogen to be moved through a cryogen flow path that includes a cryogen supply line from the cryogen source through the cryogen line 110 to the cryoprobe 114. The cryogen may then flow back to the cryogen source via a cryogen return line from the cryoprobe 114 back to the cryogen source 108.

The cryogen line 110 may be a flexible tube or other conduit that may include multiple lumens to allow the cryogen to flow in a supply direction to the cryoprobe 114 and separately in a return direction away from the cryoprobe 114. The cryogen line 110 may of sufficient length to allow the console 102 to be positioned near the patient in a treatment room and to allow the patient to be moved into and out of an imaging device. In some examples, the cryogen line may be at least about 12 feet in length. In other examples, the cryogen line 110 may have other lengths.

The cryogen line 110 fluidly couples the cryogen source 108 to the cryoprobe 114. The cryoprobe 114 may be a needle or other elongated member that is configured to be inserted into patient tissue and be positioned at or near the target tissue during treatment. The cryoprobe 114 may be configured as a cylindrical needle having an outer diameter in a range of about 1 mm to about 4 mm. The cryoprobe 114 may also include a handle 116. The handle 116 may be configured with a first portion 118 and a second portion 120. The first portion 118 may be substantially aligned with the cryogen line 110 and the second portion 120 may be offset at an angle relative to the first portion 118. The offset angle between the first portion 118 and the second portion 119 may be about 90 degrees to define a right angle handle. In other example, the first portion 118 and the second portion 120 may be offset at different angles.

In some examples, the handle 116 may include a vacuum chamber positioned at or near an outside surface of the handle 116. The vacuum chamber may insulate the exterior of the handle from the extremely low operating temperatures of the cryogen that moves through the handle to the cryoprobe 114. This may allow an operator to touch or otherwise manipulate the cryoprobe 114 during treatment.

While not shown, more than one cryoprobe assembly 112 may be coupled to the console 102. Multiple cryoprobe assemblies 112 may be used during a single cryo-procedure in combination. The console 102 may be configured to deliver cryogen to the multiple cryoprobe assemblies 112. The cryoprobes 114 of each cryoprobe assembly 112 may be similar to reach other or may be different to produce iceballs of different sizes and shapes so as to subject the neurological target tissue to the cryo-treatment zone with a lowered temperature.

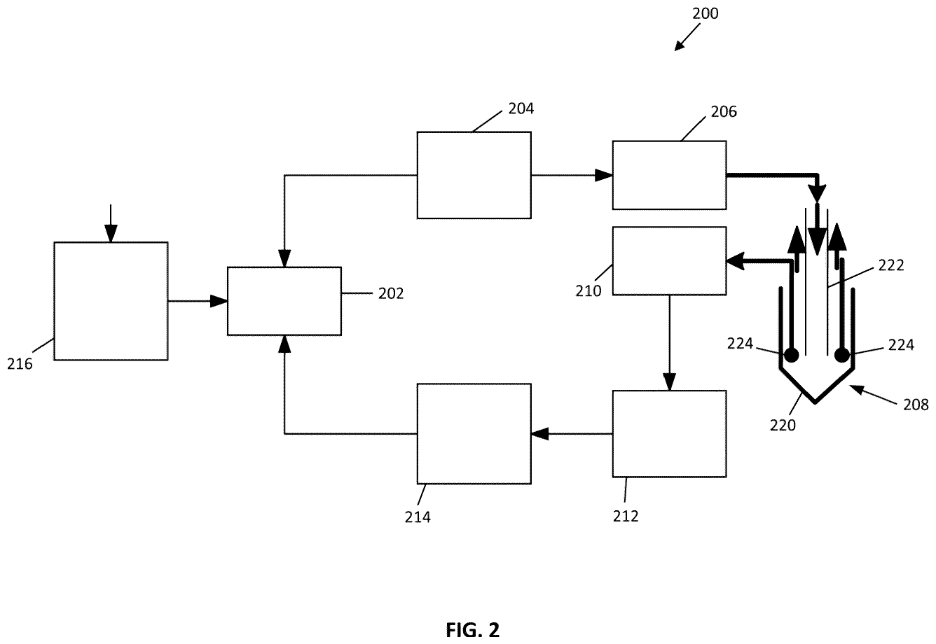

Referring now to FIG. 2, an example cryo-treatment apparatus 200 is shown. The cryo-treatment apparatus 200 may include a cryo-control 202, a cryo-model 204, a cryogen delivery module 206, a cryo-data acquisition unit 210, a cryo-data processing module 212, and a cryo-analysis module 214. While not shown, the cryo-treatment apparatus 200 may be similar to the cryo-treatment apparatus 100 previously shown. The cryo-treatment apparatus 200 may be packaged and/or structured similar to the cryo-treatment apparatus 100 to be assembled in a console or other physical unit to be used in a treatment room. In other examples, one or more of the elements of the cryo-treatment apparatus 200 may located remotely to the treatment room or may be packaged in separate physical units. In some examples, the elements of the cryo-treatment apparatus 200 may be located remotely from each other and may be connected using suitable wired or wireless communication networks.

The cryo-control 202 may include one or more computing devices that may include a processor and memory configured to perform the functions described in the present disclosure. The cryo-control 202 may include input and output devices to allow a user to input certain settings or to control or execute the functions described herein. The cryo-control 202 may be automated to perform some actions automatically with little to no input from a user.

The cryo-model 204 may be a set of executable instructions that may include one or more of algorithms, machine learning models, or the like that may provide instructions or recommendations for settings for various operating parameters of the cryo-treatment apparatus 200. The cryo-model 204, in one example, may use inputs collected from various sensors that may be located in the cryo-treatment apparatus 200, including in or on a cryoprobe 208. The cryo-model 204 may recommend or otherwise provide instructions to adjust a flow of cryogen, a pressure of cryogen, duty cycle of cryogen, a pulse of cryogen, or other functions of the cryo-treatment apparatus 200.

The cryo-model 204 may be coupled to the cryogen deliver module 206. The cryogen delivery module 206 may include one or more controllable valves, heaters, pumps or other elements that may deliver the cryogen to the cryoprobe 208. The cryo-model 204 may provide recommendations and/or instructions to the cryogen delivery module 206 that, in turn, causes a flow of cryogen to be supplied from the Dewar or other source to the cryoprobe 208 with the characteristics recommended by the cryo-model 204.

The cryogen delivery module 206 may deliver the cryogen to the cryoprobe 208 via a supply line or other conduit. The cryoprobe 208, in this example, may be configured as a Joule-Thompson cryoprobe. The cryoprobe 208 may be configured to utilize the Joule-Thompson effect to significantly decrease the operating temperature of the tip of the cryoprobe. The cryoprobe 208 may include a shell 220 configured as a needle to be inserted at or near the neurological target tissue. The shell 220 may define an internal cavity at a distal end of the cryogen supply conduit 222. When the cryogen exits the cryogen supply conduit 222 into the internal cavity, an expansion of the cryogen causes a drop in temperature due to the Joule-Thompson effect. The lower pressure cryogen may then exit the shell 220 in a cryogen return path or exhaust.

In existing Joule-Thompson cryoprobes, the cryogen is transferred to the tip of the cryoprobe at a high pressure. In some instances, the pressure may be at or above 3000 pounds per square inch (psi). Such existing Joule-Thompson cryoprobes cause a drop in temperature to operating temperatures less than −100 degrees Celsius. While such temperatures may be desirable for cryoablation, these low temperatures destroy tissue which is to be avoided in the cryo-based anesthesia apparatuses and methods of the present disclosure.

The cryoprobe 208 may also include one or more sensors 224 positioned in or on the shell 220. The sensors 224 may be pressure, impedance, and/or temperature sensors. The sensors 224 may provide cryoprobe operating information regarding operating conditions of the cryoprobe 208. The cryoprobe operating information may provide a temperature of the cryogen, a temperature of the shell 220, a pressure of the cryogen in the supply conduit 222, a pressure of the cryogen in the return path, an impedance of the tissue at the shell 220, or other measurements or data. The sensors 224 may be positioned at various locations on or in the shell 220 to provide the information at various locations along the shell 220.

The sensors 224 may be coupled to the cryo-data acquisition unit 210. The cryo-data acquisition unit 210 may be a suitable data acquisition unit and/or other device that may calculate or convert a signal received from the sensor 224 to a temperature measurement, pressure measurement, impedance measurement, flow measurement, or the like depending on the type of sensor. The cryo-data acquisition unit 210 may collect and/or store the information provided by the sensors 224.

While not shown in FIG. 2, the cryoprobe 208 may also include one or more electrical pads that may be positioned on or in the shell 220. The electrical pads may be connected to a power source in the cryo-treatment apparatus 200. The electrical pads may be configured to deliver electrical stimuli to the tissue surrounding the cryoprobe 208. Such electrical stimulation of the neurological target tissue combined with the low temperature levels may achieve masking, modulation, and pain relief to the patient. The delivery of the such electrical stimuli via the electrical pads may be controlled by the cryo-control 202.

The cryo-data acquisition unit 210 may be coupled to the cryo-data processing module 212. The cryo-data processing module 212 may perform functions to the cryoprobe operating data such as filtering, conditioning, noise-reduction, or other actions that may improve the quality of the cryoprobe operating data and allow the cryoprobe operating data to be efficiently and effectively used by the other elements of the cryo-treatment apparatus 200.

The cryo-data processing module 212 may be coupled to the cryo-analysis module 214. The cryo-analysis module 214 may perform further functions or analysis to the cryoprobe operating data that may be received from the cryo-data processing module 212. In various examples, the cryo-analysis module 214 may include statistical tools, algorithms, functions or other tools to process the cryoprobe operating data for further use. The cryo-analysis module 214 may determine an average of each measurement, a rate of change of each measurement over time, statistical parameters such as standard deviation, variation, and the like.

The cryo-analysis module 214 may be coupled to the cryo-control 202. The cryo-control 202 may also be coupled to anesthesia application module 216. The anesthesia application module 216 may be a module that supplies information to support the activities of the cryo-treatment apparatus 200. The anesthesia application module 216 may include databases and information related to clinical information about tissue types, clinical treatment plans, health information of the patient, iceball size needs, neuro conducting modulation plans and the like. The cryo-control 202 may access the information and systems of the anesthesia application module 216 and use such information as an input to the cryo-model 204 and/or to decide what changes, settings, and actions should be implemented in the cryo-treatment apparatus 200.

In some examples, the cryo-model 204 may include a machine learning model. The machine learning model may be a trained machine learning model that may be trained using historical clinical data and/or experimental data from cryo-treatments to determine an optimized operation of the cryo-treatment apparatus 200.

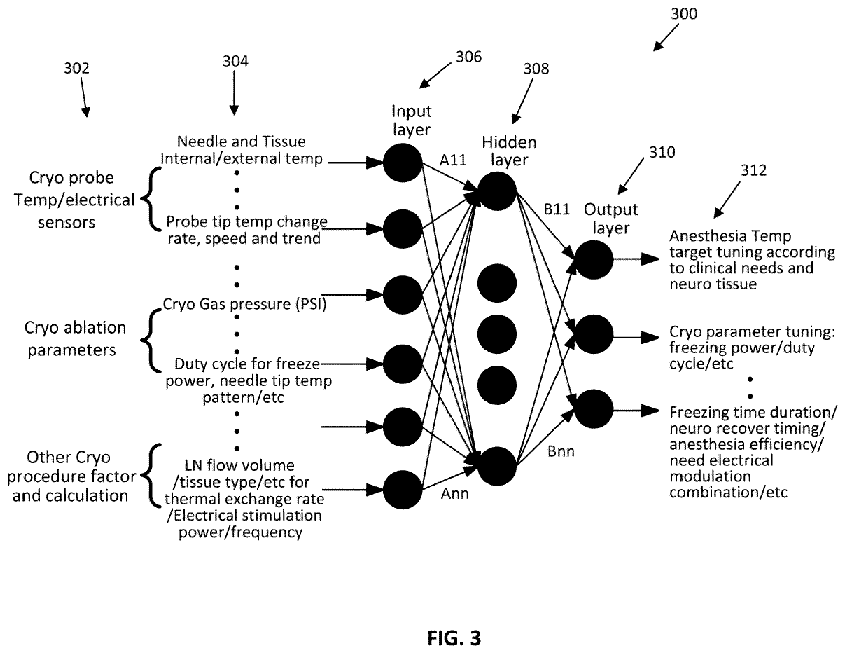

An example cryo-model 300 is shown in FIG. 3. The term model as used in the present disclosure includes data models created using machine learning and/or artificial intelligence. Machine learning may involve training a mathematical model in a supervised or unsupervised setting. Machine learning models may be trained to learn relationships between various groups of data. The models may be based on a set of algorithms that are designed to model abstractions in data by using a number of processing layers. The processing layers may be made up of non-linear transformations. Machine learning models may include, for example, neural networks, convolutional neural networks and deep neural networks. Such neural networks may be made of up of levels of trainable filters, transformations, projections, hashing, and pooling. The models may be used in large-scale relationship-recognition tasks. The models can be created by using various open-source and proprietary machine learning tools and/or libraries known to those of ordinary skill in the art.

The cryo-model 304 may use one or more data input sources 302 as inputs to the trained model. The data input sources may include cryoprobe sensors such as the sensors 224 previously described. The data input sources 302 may also include cryo-treatment parameters such as cryogen pressure, needle temperatures, duty cycles and the like. The data input sources 302 may also include other cryo-procedure factors such as cryogen flow volume, tissue type, thermal exchange rates, and electrical stimulation power and frequency.

The data input sources 302 may provide the input data 304 that may be organized or structured into a suitable data input set for supply to the model 300. The input data 304 may include, for example, needle and tissue external and internal temperatures, probe tip temperature, probe tip temperature change, probe tip rate of change, cryogen pressure, duty cycle of freeze power, needle tip temperature pattern, cryogen flow volume, tissue type, thermal exchange rate, electrical stimulation power and frequency. This information may be supplied as input data 304 to the model 304.

The model 304 may include one or more layers illustrated as an input layer 306, hidden layer 308, and output layer 310. The model 304 may use an artificial neural network or other tools to determine complex relationships among the inputs 304. The model 304 may provide outputs 312 that the cryo-control 202 may use to control, adjust, change the operating parameters of the cryo-treatment apparatus to maintain the target treatment temperature range for a predetermined period of time. The target temperature range corresponding to a temperature range to provide the cryo-based anesthesia effects previously described.

In some examples, the outputs 312 of the model 300 may provide an anesthesia target temperature, cryo-treatment apparatus parameters, such as freezing power, duty cycle, freezing time duration, neuro recovery timing, anesthesia efficiency, and/or an electrical stimulation that may accompany the freezing cycle. The outputs 312 of the model 300 may be used to optimize control of the cryo-treatment apparatus 200 during a cryo-based anesthesia treatment. The outputs 312 may be used to provide feedback to the system during an on-going cryo-treatment to adjust, change, and/or optimize the cryo-treatment.

Testing was performed using an example cryo-treatment apparatus such as the cryo-treatment apparatus 100. The cryo-treatment apparatus 100 used in the testing included multiple different Joule-Thompson cryoprobes with various needle diameters and different configurations that were developed to produced either spherical or elliptical iceball shapes. The cryogen that was used in the testing was Argon gas. The cryo-treatment apparatus was able to achieve and maintain a target treatment temperature in the range of about −60 degrees Celsius to about −100 degrees Celsius. As can be seen in the summary table below, the target treatment temperature was achieved while preventing a temperature of less than −100 degrees Celsius so as to prevent or reduce a likelihood of permanent damage to the target neurological tissue.

TABLE 1
Test Performance of Cryo-based Anesthesia

| Gas Argon | Probe Size/ | Temperature (C.) | | |
|---|---|---|---|---|
| Pressure (PSI) | Type | 3 minutes | 5 minutes | 10 minutes |
| 1500 | 2.1 mm | −35 | −52 | −65 |
| | Elliptical | | | |
| 1500 | 2.1 mm | −31 | −48 | −62 |
| | Elliptical | | | |
| 1800 | 2.1 mm | −42 | −58 | −73 |
| | Elliptical | | | |
| 1800 | 2.1 mm | −40 | −55 | −70 |
| | Spherical | | | |
| 2000 | 1.7 mm | −38 | −58 | −76 |
| | Elliptical | | | |
| 2000 | 1.7 mm | −35 | −54 | −70 |
| | Spherical | | | |
| 2500 | 1.7 mm | −45 | −71 | −95 |
| | Elliptical | | | |
| 2500 | 1.7 mm | −43 | −65 | −85 |
| | Spherical | | | |

The cryo-control 202 may select the appropriate operating parameters to achieve the desired target treatment temperature according to the clinical needs. Such clinical needs may change based on a size, location, and type of the neurological tissue and the desired modulation or interruption of pain at the neurological tissue. The cryo-control 202 may adjust a pressure of the Argon being supplied to the cryoprobe to achieve the target temperature.

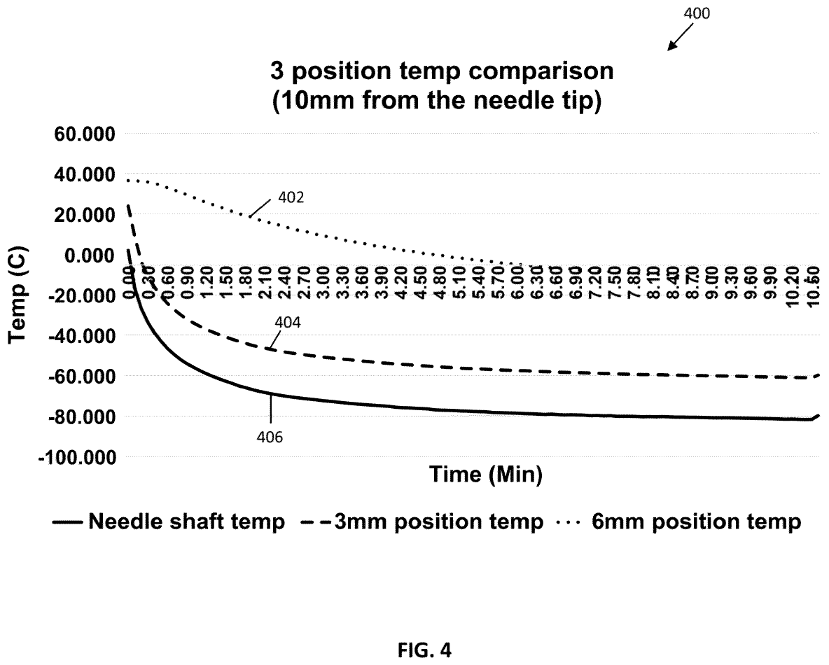

A cryo-treatment apparatus such as the cryo-treatment apparatus 100 was used to perform additional testing of a cryo-based anesthesia freezing cycle. The results of such test is shown in FIG. 4. The graph 400 illustrates a temperature that was achieved at 3 different locations in a test tissue that simulated a neurological tissue. During the testing a cryoprobe, similar to the cryoprobe 208 was used. The cryoprobe was supplied with Argon gas as the cryogen and the temperature was measured at the needle (line 406), at a position 3 mm away from the axis of the needle (line 404) and at a position 6 mm away from the axis of the needle. Each of the temperature measurements was located at an axial location about 10 mm from the tip of the cryoprobe. As shown, the cryo-treatment apparatus was able to quickly achieve a target treatment temperature of about −80 degrees Celsius at the needle. As the distance increased from the needle the temperature decreased. It is demonstrated that the cryo-treatment apparatus was able to maintain a temperature in the target temperature range during the duration of the test.

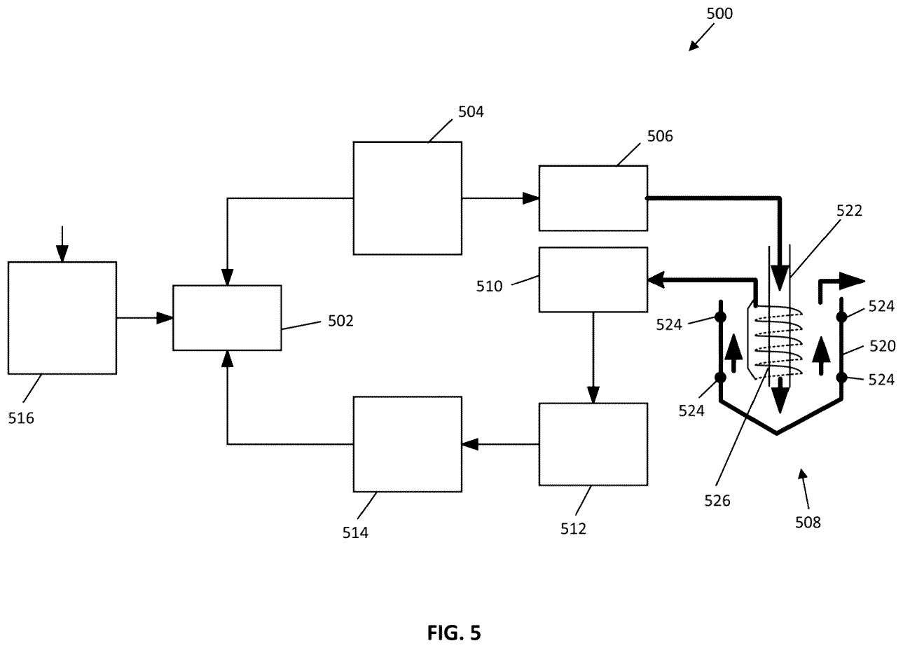

Referring now to FIG. 5, another example cryo-treatment apparatus 500 is shown. In this example, the cryo-treatment apparatus may be similar to the cryo-treatment apparatus 200 previously described. The similar elements of the cryo-treatment apparatus 500 are the cryo-control 502, the cryo-model 504, the cryo-data acquisition unit 510, the cryo-data processing module 512, and the cryo-analysis module 514. For the sake of brevity these elements are not described again but it should be appreciated that these elements may include a similar structure and perform similar functions to the corresponding elements of cryo-treatment apparatus 200.

The cryogen delivery module 506 and the cryoprobe 508 may vary from the cryogen delivery module 206 and the cryoprobe 208 previously described. In this example, the cryoprobe 508 may use a liquid cryogen rather than the gaseous cryogen previously described. The cryoprobe 508 may not be a Joule-Thompson probe but may use a liquid cryogen such as liquid Nitrogen for the cooling the probe. The use of liquid Nitrogen may provide an advantage over gas cryogen (e.g., gas Argon) because liquid Nitrogen may provide faster freezing than gas Argon. Liquid Nitrogen may also be less expensive than Argon. The use of liquid Nitrogen, however, may require the use of a heater 526 in the cryoprobe in order to achieve the target treatment temperatures for cryo-based anesthesia.

The cryoprobe 508 may include a shell 520 that forms a needle that terminates at a tip of the cryoprobe 508. The shell 520 may be an outer wall that defines an inner cavity in which various elements of the cryoprobe 508 may be positioned. A cryogen supply 522 may form a supply path for the cryogen to be moved into the cryoprobe 508 toward the tip of the shell 520. The cryogen may then flow away from the tip in a cryogen return defined as the space between the outer surface of the cryogen supply 522 and the inner surface of the shell 520. This flow of cryogen may cause the cryoprobe to lower the temperature of the target neurological tissue and cause an iceball to form at the target neurological tissue.

The cryoprobe 508 may also include a heater 526 that is positioned inside the shell 520 or on the shell 520. The heater 526, in one example, may be a resistive heater that is coupled to a suitable power source that when energized heats the cryoprobe 508. The heater 526 may be energized during a cryo-based anesthesia treatment to moderate the temperature of the cryoprobe 508. Because liquid Nitrogen has such a low temperature, the heater 526 may be required to maintain the cryoprobe 508 and the surrounding target neurological tissue at the desired target temperature range. Without the heater 526, the cryoprobe 508 may drop to temperatures at which the target neurological tissue may be permanently damaged or destroyed. With the use of liquid Nitrogen and the heater 526, the temperature of the target neurological tissue can be maintained in a range of about −50 degrees Celsius to about −100 degrees Celsius.

The cryoprobe 508 may also include one or more sensors 524. The sensors may be similar to the sensors 224. The sensors 524 may be positioned at various locations in the shell 520 or on the shell 520. The sensors 524 may provide cryoprobe operating information to the cryo-control 502. The sensors 524 may be thermocouples, temperature sensors, pressure sensors, impedance sensors, flow sensors or other sensors to characterize operating conditions of the cryo-treatment apparatus 500. The sensors 524 may be coupled to the cryo-control 502 and/or to the cryo-data acquisition unit 510 via suitable wired or wireless connections.

While not shown in FIG. 5, the cryoprobe 508 may also include one or more electrical pads that may be positioned on or in the shell 520. The electrical pads may be connected to a power source in the cryo-treatment apparatus 500. The electrical pads may be configured to deliver electrical stimuli to the tissue surrounding the cryoprobe 508. Such electrical stimulation of the neurological target tissue combined with the low temperature levels may achieve masking, modulation, and pain relief to the patient. The delivery of the such electrical stimuli via the electrical pads may be controlled by the cryo-control 502.

In some examples, the cryo-model 504 may include a machine learning model. The machine learning model may be a trained machine learning model that may be trained using historical clinical data and/or experimental data from cryo-treatments to determine an optimized operation of the cryo-treatment apparatus 500.

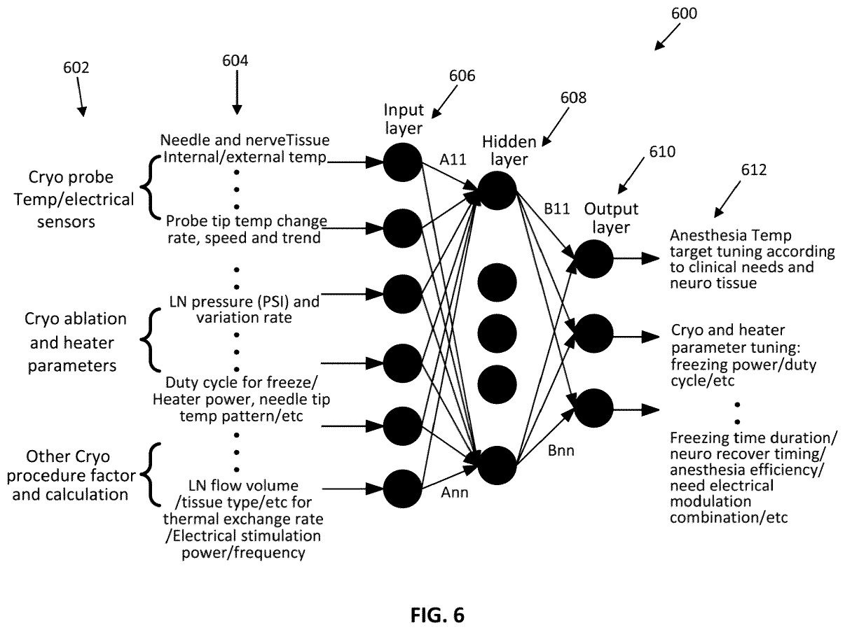

An example cryo-model 600 is shown in FIG. 6. The cryo-model 600 may be similar to the cryo-model 300 previously described. The model 600 may be trained to learn relationships between various groups of data. The models may be based on a set of algorithms that are designed to model abstractions in data by using a number of processing layers. The processing layers may be made up of non-linear transformations. Machine learning models may include, for example, neural networks, convolutional neural networks and deep neural networks. Such neural networks may be made of up of levels of trainable filters, transformations, projections, hashing, and pooling. The model 600 may be used in large-scale relationship-recognition tasks. The model 600 can be created by using various open-source and proprietary machine learning tools and/or libraries known to those of ordinary skill in the art.

The cryo-model 504 may use one or more data input sources 602 as inputs to the trained model. The data input sources may include cryoprobe sensors such as the sensors 524 previously described. The data input sources 602 may also include cryo-treatment parameters such as heater temperature, heater power or duty cycle, cryogen pressure, needle temperatures, duty cycles and the like. The data input sources 602 may also include other cryo-procedure factors such as cryogen flow volume, tissue type, thermal exchange rates, and electrical stimulation power and frequency.

The data input sources 602 may provide the input data 604 that may be organized or structured into a suitable data input set for supply to the model 600. The input data 604 may include, for example, needle and tissue external and internal temperatures, probe tip temperature, probe tip temperature change, probe tip rate of change, cryogen pressure, heater temperature, heater power, heater duty cycle, duty cycle of freeze power, needle tip temperature pattern, cryogen flow volume, tissue type, thermal exchange rate, electrical stimulation power and frequency. This information may be supplied as input data 604 to the model 604.

The model 604 may include one or more layers illustrated as an input layer 606, hidden layer 608, and output layer 610. The model 604 may use an artificial neural network or other tools to determine complex relationships among the inputs 604. The model 604 may provide outputs 612 that the cryo-control 502 may use to control, adjust, change the operating parameters of the cryo-treatment apparatus to maintain the target treatment temperature range for a predetermined period of time. The target temperature range corresponding to a temperature range to provide the cryo-based anesthesia effects previously described.

In some examples, the outputs 612 of the model 600 may provide an anesthesia target temperature, cryo-treatment apparatus parameters, such as freezing power, heater power, heater timing, heater, modulation, duty cycle, freezing time duration, neuro recovery timing, anesthesia efficiency, and/or an electrical stimulation that may accompany the freezing cycle. The outputs 612 of the model 600 may be used to optimize control of the cryo-treatment apparatus 500 during a cryo-based anesthesia treatment. The outputs 612 may be used to provide feedback to the system during an on-going cryo-treatment to adjust, change, and/or optimize the cryo-treatment.

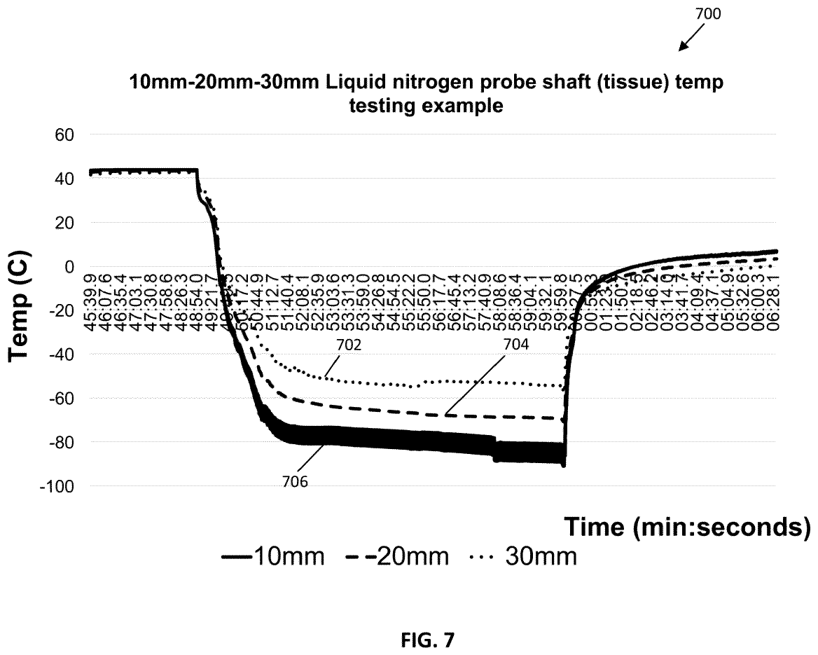

Testing was performed using a cryo-treatment apparatus similar to the cryo-treatment apparatus 500. The testing used a cryoprobe, such as cryoprobe 508, that includes a supply of liquid Nitrogen and a heater. The test cryoprobe was position in an analogue material to simulate a neurological tissue. A temperature along the axis of the cryoprobe was measured at various locations relative to the probe tip. As shown in FIG. 7, the temperature at 10 mm from the probe tip (line 706), a temperature at 20 mm from the probe tip (line 704) and a temperature at 30 mm from the probe tip were recorded during a cryo-based anesthesia cycle. The cryo-treatment apparatus was operated to target a temperature of −90 degrees Celsius at the probe for a period of approximately 10 minutes. As can be seen, the temperature was maintained at the desired temperature and no locations at the cryoprobe exceeded a temperature of −100 degrees Celsius in order to operate the apparatus to prevent or minimize a risk of permanent damage to a neurological tissue.

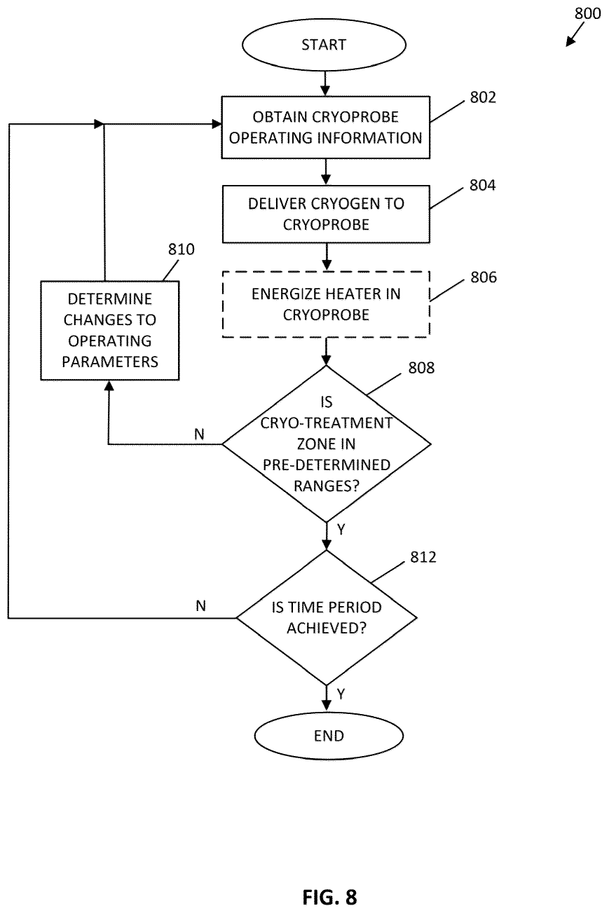

Referring now to FIG. 8, an example method 800 of cryo-based anesthesia is shown. The method 800 may be performed using one of the cryo-treatment apparatuses of the present disclosure. For illustration purposes the method 800 is described below as being performed by the cryo-treatment apparatuses 200 or 500 but it should be appreciated that other apparatuses may be used to perform the method.

The method 800 may start at step 802. At step 802, the cryo-data acquisition unit 210, 510 may obtain cryoprobe operating information. The cryoprobe operating information may be obtained from one or more sensors. The cryoprobe operating information may include information regarding a temperature, pressure, flow, or other characteristic of the cryogen in the cryoprobe. The cryoprobe operating information may also include one or more characteristics of the tissue that may be positioned at or around the cryoprobe such as a tissue type or measured impedance at the cryoprobe. In still other examples, the cryoprobe operating information may include the data described as inputs to the cryo-model 300, 600. The cryoprobe operating information may be collected by the sensors 224, 524 previously described.

At step 804, the cryo-control may deliver cryogen to the cryoprobe. The cryo-control may cause the cryogen delivery module to deliver the cryogen to the cryoprobe. One or more pumps, valves or other aspects of the cryogen delivery module may be activated to cause the cryogen to flow to the cryoprobe. The delivery of the cryogen to the cryoprobe causes the temperature of the cryoprobe to drop and to remove heat from the target neurological tissue and to cause an iceball to grow. The cryogen may be delivered at a predetermined pressure and/or temperature at step 804 to achieve desired characteristics of the cryo-treatment zone at the target tissue. Such characteristics may include a treatment temperature range and/or a size of an iceball. As compared to cryoablation, the size of an iceball for cryo-based anesthesia may be significantly smaller. In various examples, the target size of the iceball in cryo-based anesthesia may be less than about 2 cm.

Step 806 is an optional step that may be used particularly with respect to cryo-treatment apparatuses that use liquid Nitrogen as the cryogen. In such systems, the temperature of the liquid Nitrogen may be at a low temperature such that the cryo-control energizes a heater in the cryoprobe to moderate the temperature drop. In Joule-Thompson systems, step 806 may not be performed and the temperature of the cryo-treatment zone may be controlled via the pressure of the cryogen.

At step 808, the cryo-control may determine whether the cryo-treatment zone is in the predetermined ranges. The cryo-control may use the cryoprobe operating information that is obtained at step 802 and compare such operating information to the predetermined ranges. Such predetermined ranges may include a treatment temperature range for the cryo-base anesthesia treatment. The treatment temperature range may be a range of about −50 degrees Celsius to about −100 degrees Celsius.

The cryo-control may prevent the temperature of the cryo-treatment zone to drop below −100 degrees Celsius to prevent permanent damage to the neurological tissue. In other examples, the type of target neurological tissue or location of the target neurological tissue may cause other target temperature ranges to be used. Such other ranges may include a range of about −50 degrees Celsius to about −80 degrees Celsius. The cryo-control may also compare pressure ranges to target pressure ranges, compare flows to target flow ranges, and other target ranges.

If the cryo-control determines that the cryo-treatment zone has characteristics in the pre-determined target ranges, the method 800 may proceed to step 812. If the cryo-control determines that the cryo-treatment zone is not in the predetermined target ranges, the method 800 may proceed to step 810.

At step 810, the cryo-control may determine changes to the cryoprobe operating parameters. The cryo-control may, for example, change an operating parameter to raise or lower a temperature of the cryo-treatment zone. The cryo-control may determine that a pressure of the cryogen may need to be increased or may determine that a power supplied to the heater may need to be increased. Alternatively, the cryo-control may determine that a pressure of the cryogen may need to be decreased or may determine that a power supplied to the heater may need to be decreased. The cryo-control may use the cryo-model to determine what actions need to be taken in various examples. The cryoprobe operating information may be used as an input to determine a target operating range for one or more aspects of the cryo-treatment apparatus.

After determining (and implementing) the adjustments or changes to the cryo-treatment apparatus, the method 800 may return to step 802 to re-perform steps 802 to 808. In such a manner, the cryo-treatment apparatus may monitor and adjust the operating parameters of the cryo-treatment apparatus to maintain the cryo-treatment zone with the desired characteristics to achieve the cryo-based anesthesia of the target neurological tissue. This is different and an improvement over known cryoablation apparatuses and methods that typically seek to achieve the fastest temperature reduction and iceball growth to destroy a target tissue. The methods and apparatuses of the present disclosure seek to moderate such low temperatures and to achieve low temperatures that do not cause permanent damage to neurological tissue.

Ate step 812, the cryo-control may determine whether the target time period has been achieved. To achieve the cryo-based anesthesia, the cryo-treatment zone is maintained within the predetermined ranges for a predetermined period of time. Such target time periods may vary depending on the type of tissue and the desired anesthesia effect. In some examples, the target time period is a time of about 3 minutes to about 10 minutes. In other examples, other time periods may be used.

If the cryo-control determines that the target time period has been achieved, the method 800 may end. If the cryo-control determines that the target time period has not been achieve, the method 800 may return to step 802 and re-perform steps 802 to 812. In this manner, the cryo-treatment apparatus may maintain the cryo-treatment zone in the target ranges for the predetermined period of time.

The following is a list of non-limiting illustrative embodiments disclosed herein:

Illustrative embodiment 1: A cryo-treatment apparatus comprising: a cryoprobe assembly comprising a needle for insertion at a target tissue; a cryogen delivery apparatus fluidly connected to the cryoprobe assembly; and a cryo-treatment control apparatus comprising at least one processor and memory, wherein the cryo-treatment control apparatus is configured to: obtain cryoprobe operating information from one or more sensors positioned in the needle of the cryoprobe, the cryoprobe operating information characterizing one or more operating parameters of the cryoprobe; deliver a cryogen to the cryoprobe to produce a cryo-treatment zone in an area of the target tissue, the cryo-treatment zone having a treatment temperature range of about −50 degrees C. to about −100 degrees C.; and maintain the cryo-treatment zone in the treatment temperature range for a predetermined period of time.

Illustrative embodiment 2: The cryo-treatment apparatus of illustrative embodiment 1, wherein the needle defines a Joule-Thompson expansion chamber at a distal end thereof.

Illustrative embodiment 3: The cryo-treatment apparatus of any of illustrative embodiments 1 or 2, wherein the cryogen delivery apparatus comprises a Dewar and a pump configured to deliver the cryogen to the cryoprobe assembly.

Illustrative embodiment 4: The cryo-treatment apparatus of any of illustrative embodiments 1 to 3, wherein the cryo-treatment zone is maintained in the treatment temperature range by adjusting a pressure of the cryogen delivered to the needle.

Illustrative embodiment 5: The cryo-treatment apparatus of any of illustrative embodiments 1 to 4, wherein the cryogen comprises Argon.

Illustrative embodiment 6: The cryo-treatment apparatus of illustrative embodiment 1, wherein the needle comprises a heater and the cryo-treatment control apparatus is configured to energize and de-energize the heater to maintain the cryo-treatment zone in the treatment temperature range.

Illustrative embodiment 7: The cryo-treatment apparatus of any of illustrative embodiments 5 or 6, wherein the cryogen comprises liquid Nitrogen.

Illustrative embodiment 8: The cryo-treatment apparatus of any of illustrative embodiments 1 to 6, wherein the step of delivering the cryogen to the cryoprobe comprises pulsing the cryogen.

Illustrative embodiment 9: The cryo-treatment apparatus of any of illustrative embodiments 1 to 8, wherein the target tissue comprises a neurological tissue.

Illustrative embodiment 10: The cryo-treatment apparatus of any of illustrative embodiments 1 to 9, wherein the cryo-treatment zone changes a function of the target tissue but does not permanently damage the target tissue.

Illustrative embodiment 11: The cryo-treatment apparatus of any of illustrative embodiments 1 to 10, wherein the cryo-treatment zone is maintained in the treatment temperature range by adjusting a pressure of the cryogen delivered to the needle and adjusting a power signal delivered to a heater in the needle.

Illustrative embodiment 12: The cryo-treatment apparatus of any of illustrative embodiments 1 to 11, wherein the delivery of the cryogen to the cryoprobe causes an iceball to form at the target tissue, a desired diameter of the iceball being less than 2 cm.

Illustrative embodiment 13: The cryo-treatment apparatus of any of illustrative embodiments 1 to 12, wherein the needle comprises a conductive tip coupled to a power source for electrical stimulation of target tissue.

Illustrative embodiment 14: The cryo-treatment apparatus of any of illustrative embodiments 1 to 13, wherein the cryo-treatment control apparatus comprises a trained machine learning model configured to control at least one of a supply valve, a supply pump, a heater in a Dewar, and a heater in the needle.

Illustrative embodiment 15: The cryo-treatment apparatus of any of illustrative embodiments 1 to 14, wherein one or more sensors are positioned on or in the heater in the needle.

Illustrative embodiment 16: The cryo-treatment apparatus of any of illustrative embodiments 14 or 15, wherein the cryogen comprises liquid Nitrogen.

Illustrative embodiment 17: The cryo-treatment apparatus of illustrative embodiments 14 or 15, wherein the cryogen comprises Argon.

Illustrative embodiment 18: A method comprising: obtaining, via a cryo-treatment control apparatus comprising at least one processor and memory, cryoprobe operating information from one or more sensors positioned in a needle of a cryoprobe, the cryoprobe operating information characterizing one or more operating parameters of the cryoprobe; delivering, via a cryogen delivery apparatus coupled to the cryo-treatment control apparatus, a cryogen to the cryoprobe to produce a cryo-treatment zone in an area of the target tissue, the cryo-treatment zone having a treatment temperature range of about −50 degrees C. to about −100 degrees C.; and maintaining, via the cryogen delivery apparatus and the cryo-treatment control apparatus, the cryo-treatment zone in the treatment temperature range for a predetermined period of time.

Illustrative embodiment 19: The method of illustrative embodiment 18, wherein the needle defines a Joule-Thompson expansion chamber at a distal end thereof and the cryo-treatment zone is maintained in the treatment temperature range by adjusting a pressure of the cryogen delivered to the needle.

Illustrative embodiment 20: The method of any of illustrative embodiments 18 or 19, wherein the cryogen delivery apparatus comprises a Dewar and a pump configured to deliver liquid nitrogen to the cryoprobe assembly and the cryo-treatment zone is maintained in the treatment temperature range by adjusting a pressure of the cryogen delivered to the needle and adjusting a power signal delivered to a heater in the needle.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A cryo-treatment apparatus comprising:
a cryoprobe assembly comprising a needle for insertion at a target tissue;
a cryogen delivery apparatus fluidly connected to the cryoprobe assembly; and
a cryo-treatment control apparatus comprising at least one processor and memory, wherein the cryo-treatment control apparatus is configured to:
obtain cryoprobe operating information from one or more sensors positioned in the needle of the cryoprobe, the cryoprobe operating information characterizing one or more operating parameters of the cryoprobe;
deliver a cryogen to the cryoprobe to produce a cryo-treatment zone in an area of the target tissue, the cryo-treatment zone having a predetermined treatment temperature range; and
maintain the cryo-treatment zone in the treatment temperature range for a predetermined period of time.

2. The cryo-treatment apparatus of claim 1, wherein the predetermined treatment temperature range is maintained in a range of about −50 degrees Celsius to about −100 degrees Celsius and the predetermined period of time is between about 3 minutes to about 10 minutes.

3. The cryo-treatment apparatus of claim 1, wherein the needle defines a Joule-Thompson expansion chamber at a distal end thereof.

4. The cryo-treatment apparatus of claim 3, wherein the cryogen delivery apparatus comprises a Dewar and a pump configured to deliver the cryogen to the cryoprobe assembly.

5. The cryo-treatment apparatus of claim 3, wherein the cryo-treatment zone is maintained in the treatment temperature range by adjusting a pressure of the cryogen delivered to the needle.

6. The cryo-treatment apparatus of claim 5, wherein the cryogen comprises Argon.

7. The cryo-treatment apparatus of claim 1, wherein the needle comprises a heater and the cryo-treatment control apparatus is configured to energize and de-energize the heater to maintain the cryo-treatment zone in the treatment temperature range.

8. The cryo-treatment apparatus of claim 7, wherein the cryogen comprises liquid Nitrogen.

9. The cryo-treatment apparatus of claim 1, wherein the step of delivering the cryogen to the cryoprobe comprises pulsing the cryogen.

10. The cryo-treatment apparatus of claim 1, wherein the target tissue comprises a neurological tissue.

11. The cryo-treatment apparatus of claim 1, wherein the cryo-treatment zone changes a function of the target tissue but does not permanently damage the target tissue.

12. The cryo-treatment apparatus of claim 1, wherein the cryo-treatment zone is maintained in the treatment temperature range by adjusting a pressure of the cryogen delivered to the needle and adjusting a power signal delivered to a heater in the needle.

13. The cryo-treatment apparatus of claim 1, wherein the delivery of the cryogen to the cryoprobe causes an iceball to form at the target tissue, a desired diameter of the iceball being less than 2 cm.

14. The cryo-treatment apparatus of claim 1, wherein the needle comprises a conductive tip coupled to a power source for electrical stimulation of target tissue.

15. The cryo-treatment apparatus of claim 1, wherein the cryo-treatment control apparatus comprises a trained machine learning model configured to control at least one of a supply valve, a supply pump, a heater in a Dewar, and a heater in the needle.

16. The cryo-treatment apparatus of claim 1, wherein the cryogen comprises liquid Nitrogen.

17. The cryo-treatment apparatus of claim 1, wherein the cryogen comprises Argon.

18. A method comprising:
obtaining, via a cryo-treatment control apparatus comprising at least one processor and memory, cryoprobe operating information from one or more sensors positioned in a needle of a cryoprobe, the cryoprobe operating information characterizing one or more operating parameters of the cryoprobe;
delivering, via a cryogen delivery apparatus coupled to the cryo-treatment control apparatus, a cryogen to the cryoprobe to produce a cryo-treatment zone in an area of the target tissue, the cryo-treatment zone having a treatment temperature range of about −50 degrees C. to about −100 degrees C.; and
maintaining, via the cryogen delivery apparatus and the cryo-treatment control apparatus, the cryo-treatment zone in the treatment temperature range for a predetermined period of time.

19. The method of claim 18, wherein the needle defines a Joule-Thompson expansion chamber at a distal end thereof and the cryo-treatment zone is maintained in the treatment temperature range by adjusting a pressure of the cryogen delivered to the needle.

20. The method of claim 18, wherein the cryogen delivery apparatus comprises a Dewar and a pump configured to deliver liquid nitrogen to the cryoprobe assembly and the cryo-treatment zone is maintained in the treatment temperature range by adjusting a pressure of the cryogen delivered to the needle and adjusting a power signal delivered to a heater in the needle.
